(19) United States
(12) Patent Application Publication
(10) Pub. No.: US 2014/0180073 A1
(43) Pub. Date: Jun. 26, 2014

(54) PORTABLE DIGITAL IMAGING SYSTEM FOR FLUORESCENCE-GUIDED SURGERY

(71) Applicant: AntiCancer, Inc., San Diego, CA (US)

(72) Inventors: Yukihiko Hiroshima, San Diego, AZ (US); Yong Zhang, San Diego, AZ (US)

(73) Assignee: AntiCancer, Inc., San Diego, AZ (US)

(21) Appl. No.: 14/140,333

(22) Filed: Dec. 24, 2013

Related U.S. Application Data
(60) Provisional application No. 61/745,730, filed on Dec. 24, 2012.

(51) Int. Cl. A61B 5/06; A61B 5/00

(52) U.S. Cl. CPC A61B 5/061; A61B 5/0071
USPC 600/424

(57) ABSTRACT

A portable digital imaging system for fluorescence-guided surgery is provided. The system includes a portable probe comprising a light source, a fluorescence detector, a digital signal output, a memory and a processor. The system also includes a computer in communication with the portable probe and a display. The portable probe excites a fluorescent label-containing tissue within a surgical field in response to the light source of the portable probe illuminating the fluorescent label-containing tissue. The computer displays a real-time image visualization of the surgical field on the display in response to the computer in communication with the display receiving and processing a digital signal from the digital signal output of the portable probe.

### CROSS REFERENCE TO RELATED APPLICATION

This application claims priority from U.S. Patent Application No. 61/745,730, filed Dec. 24, 2012 and entitled “Portable Digital Imaging System for Fluorescence-Guided Surgery (FGS),” which is incorporated entirely herein by reference.

### BACKGROUND OF THE INVENTION

1. Technical Field

This invention relates to the use of a portable florescence imaging device for florescence-guided surgery (“FGS”). In particular, the invention involves the use of a portable digital florescence imaging device in the florescent-guided surgery of cancer.

2. State of the Art

The long-term survival of patients with solid tumor malignancies is typically dependent on complete surgical resection of the primary tumor. Additionally, accurate staging to determine an optimal strategy for post-surgical adjuvant treatment depends on the early identification of the presence and extent of metastatic disease. Therefore, complete visualization of primary and metastatic tumors within the operative field at the time of surgery is integral to the treatment of patients with solid cancers.

Fluorescence-guided surgery enhances tumor identification and resection. Viewing of tumors glowing with fluorescence under excitatory light is a powerful adjunct to intraoperative tumor visualization over standard “bright light” surgery (“BLS”). Labeling of primary and metastatic solid tumors using intracellular fluorescent labels has been accomplished through a variety of techniques, including retroviral transfection of genes coding for fluorescent proteins, fluorophores, and fluorescent antibodies. The label allows for greatly increased visual resolution when excited to fluoresce by a light source of the proper wavelength. Resolution is further increased by converting the emitted fluorescent light to a digital signal that can be viewed on a computer screen or other standard monitor. Fixed fluorescence imaging systems are typically used for this purpose.

These fixed fluorescence imaging systems, although easily used in the laboratory for FGS on small experimental animals, are impractical for use in the hospital operating room and other clinical settings when treating human cancer patients. For example, the Olympus OV-100 small animal imaging system for in-vivo fluorescence molecular imaging is contained within a 50 cm×54 cm×100 cm. cabinet and requires introducing the entire animal into the device, yet has a field of view no greater than 6.3 cm. at a magnification of 0.14×. (http://www.metamouse.com/OV100%20brochure.pdf) Such a device cannot be used by the gowned-and-gloved surgeon, who ideally must be able to direct the device to the area of interest within the surgical field without breaking sterility. Intraoperative use by the surgeon is necessary to facilitate complete resection of the primary tumor and metastatic deposits while optimizing preservation of normal, non-cancerous tissue. Additionally, fixed imaging systems can induce significant background autofluorescence of non-labeled tissue, decreasing resolution and creating ambiguity regarding the boundary between labeled and non-labeled tissue. The value of using fluorescent labels lies in making this precise distinction. For example, to optimize the chance of curing a patient with a solid-tumor malignancy, all tissue containing cancerous cells must be resected while minimizing excision of non-labeled normal somatic cells to preserve the function of normal tissues and organs.

In order for FGS to become routine in clinical settings and to enhance its use in the laboratory, an improved imaging system is needed.

Citation of documents herein is not an admission by the applicant that any is pertinent prior art. Stated dates or representation of the contents of any document is based on the information available to the applicant and does not constitute any admission of the correctness of the dates or contents of any document.

### SUMMARY OF THE INVENTION

Embodiments of the present invention relate to a system and method for using a portable digital fluorescence imaging device for fluorescence-guided surgery and for fluorescence-guided surgery of cancer. Generally, the system and method include the use of a portable probe directed toward a surgical field to excite a fluorescent label-containing tissue within the surgical field and display a real-time image visualization of the surgical field on a display.

An embodiment includes a method for use of a portable imaging device for fluorescence-guided surgery. The method comprises directing a portable probe toward a surgical field, wherein the probe comprises a light source, a fluorescence detector, and a digital signal output. The method also includes exciting a fluorescent label-containing tissue within the surgical field in response to the light source of the portable probe illuminating the fluorescent label-containing tissue. The method then includes displaying a real-time image visualization of the surgical field on a screen in response to a computer in communication with the computer receiving image data of the fluorescent label-containing tissue from the digital signal output of the portable probe.

It is understood that where “screen,” “monitor,” or other related term is used throughout this application, embodiments of the invention may use these or any type and number of displays for visualization during FGS.

Another embodiment includes a portable digital imaging system for fluorescence-guided surgery. The system comprises a portable probe comprising a light source, a fluorescence detector, a digital signal output, a memory and a processor. The system also includes a computer in communication with the portable probe, the computer comprising a memory and a processor, and a display. The portable probe excites a fluorescent label-containing tissue within a surgical field in response to the light source of the portable probe illuminating the fluorescent label-containing tissue. Further, the computer displays a real-time image visualization of the surgical field on the screen in response to the computer receiving and processing a digital signal from the output of the portable probe.

The foregoing and other features and advantages of the present invention will be apparent from the following more detailed description of the particular embodiments of the invention, as illustrated in the accompanying drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic of an example handheld probe for real-time digital imaging of tissues containing fluorescent-labeled cells.

FIG. 2 is a flowchart demonstrating the steps to practice the invention.

FIG. 3 is a series of photographs demonstrating an example portable imaging system for use in FGS, contrasted with two corresponding fixed imaging systems.

FIG. 4 is imaging of genetically-labeled MiaPaCa-2-GFP tumor using an example portable imaging system for use in FGS, contrasted with images taken with two corresponding fixed imaging systems.

FIG. 5 is imaging of the BxPC3 tumor labeled with fluorescent anti-CEA antibody using an example portable imaging system for use in FGS, contrasted with images taken with two corresponding fixed imaging systems.

FIG. 6 is imaging of the pancreatic PDOX® tumor labeled with fluorescent anti-CA19-9 antibody using an example portable imaging system for use in FGS contrasted with images taken with two corresponding fixed imaging systems

### DETAILED DESCRIPTION OF EMBODIMENTS OF THE INVENTION

Since the discovery of vaccines and antibiotics to prevent and treat infectious disease, cancer has become a leading cause of death in adults over the age of forty-five. Only heart disease is responsible for more deaths annually. Most cell types, including the most common varieties causing death—lung, breast, colon, and pancreas, initially form a solid primary tumor. If this tumor is not treated, the disease progresses. A growing, invasive primary tumor compresses and invades adjacent vital structures, interfering with their function. Individual cancer cells separate from the primary tumor and metastasize via lymphatics to regional lymph nodes, or hematogenously to distant organs, commonly the lungs, liver, brain, and bone marrow.

In the overwhelming majority of cancers forming solid tumors, complete surgical resection of all cancerous cells affords the only chance for cure. A complete resection requires obtaining a cancer cell-free margin around the entire periphery of the resected primary tumor. It also requires identification and typically resection of secondary metastatic tumors. In both the resection of the primary tumor and the identification and resection of metastases, the surgeon is limited by unaided senses of sight and touch when defining the precise location and extent of both the primary tumor and any regional metastatic disease within the surgical field. The human physical senses, however, are unable to determine the completeness of resection with sufficient precision. Thus, the ability of the surgeon to accurately define tumor margins, visualize any residual tumor in the wound bed, and identify metastases at the time of surgery can determine success of cancer surgery. In the operating room, adjuncts to the senses are needed.

Diagnostic imaging modalities (examples include x-ray fluoroscopy, plain portable x-rays films, and intraoperative ultrasound) have long been used during surgical exploration as adjuncts to determine the extent of disease and guide the surgical resection. Although useful in defining the proximity and relationship of the tumor to surrounding vital structures, these modalities cannot provide direct, real-time visualization of the extent of the primary tumor or regional metastatic disease. Rather, they indicate only the gross extent of the tumor mass without great precision. The surgeon is left to make a ballpark assessment of where the tumor(s) end and non-cancerous tissue begins.

Frozen-section histological examination of resected tumors to confirm the absence of tumor cells at the surgical margin—an uninterrupted rim of non-cancerous cells surrounding the entire periphery of the resected specimen suggesting complete resection—is an established and widely-available technique. This examination is done during the operation, but the tissue to be examined is removed from the operating room to a separate location in the hospital. Unlike the intraoperative imaging modalities mentioned above, this technique is able to identify a single cancer cell at the margin of the section with microscopic precision, implying an incomplete resection. Again, however, the choice of tissue resected for examination is determined by the surgeon's unaided senses of sight and touch. Additionally, evaluation of surgical margins by frozen section is limited by the number of sections examined and the location on the resected specimen from which the sections are prepared for microscopic examination by the pathologist. The pathologist, and not the surgeon, determines exactly where on the specimen to take the section(s). The technique's accuracy is, therefore, subject to sampling error. Finally, the surgeon generally must depend on an accurate interpretation of the microscopic histopathology viewed by the examining pathologist, rather than making a direct assessment.

None of these modalities provide enhanced direct, high-resolution real-time intraoperative visualization of the cancerous or other tissue of interest by the operating surgeon.

Fluorescence imaging has been shown to be optimal for cancer navigation and offers higher resolution and sensitivity compared to radiological imaging and to visual bright-light inspection and palpation during surgery. (Van Dam et al. Intraoperative tumor-specific fluorescence imaging in ovarian cancer by folate receptor-α targeting: first in-human results. Nat. Med. 17, 1315-19 (2011).) Negative surgical margins are vital to achieve cure and prolong survival in patients with cancer. FGS improves surgical outcomes and reduces recurrence rates in an orthotopic mouse model of human pancreatic cancer. (Metildi, C. C. et al. Fluorescence-guided surgery allows for more complete resection of pancreatic cancer, resulting in longer disease-free survival compared with standard surgery in orthotopic mouse models. J. Am. Coll. Surg. 215, 126-35 (2012)), and colon cancer. (Metildi, C. A. et al. Fluorescence-guided surgery of human colon cancer increases complete resection resulting in cures in an orthotopic nude mouse model. J. Surg. Res. 179, 87-93 (2013).)

Fluorescent labels, including chromophore molecules, fluorescent proteins, and fluorescent antibodies have all been used to label individual cells. A predominant fluorescent protein is green fluorescent protein (“GFP”). GFP and related fluorescent proteins are a homologous protein family, having emission spectra ranging from 442 to 645 nm or longer. They range in size from 25 to 30 kDa and form internal chromophores that do not require cofactors or substrates to fluoresce. These fluorescent proteins have very high extinction coefficients ranging from E=6,500 up to E=95,000. In addition, they have very high quantum yields ranging from 0.24 up to 0.8. These properties make fluorescent proteins very bright. The large two-photon absorption of GFP is important for in vivo applications. Another important feature is the spectral distinction between many members of the family, so a set of multicolor fluorescent proteins can be used simultaneously for multifunctional in vivo imaging. All of these combined properties make fluorescent proteins optimal for cellular imaging in vivo. Cells labeled with GFP glow very strongly when exposed to light of an appropriate excitatory wavelength within the blue range of the visible spectrum. (Hoffman, R. M. The multiple uses of fluorescent proteins to visualize cancer in vivo. Nature Reviews Cancer 5, 796-806 (2005).) Fluorescent labeling with GFP has important advantages over other optical imaging methods in vivo. GFP has a very strong signal and images can be captured with fairly simple apparatus with no need for total darkness. Fluorescent proteins cone in many colors, allowing for multiple events to be imaged. Additionally, GFP fluorescence is relatively unaffected by the external environment, as the chromophore is protected by the three-dimensional structure of the protein. (Hoffman, R. M. The multiple uses of fluorescent proteins to visualize cancer in vivo. Nature Reviews Cancer 5, 796-806 (2005).)

Preclinically, the most promising tumor-labeling methods for FGS exploit tumor-specific retroviruses to transfect tumor cells with the GFP gene, or utilize tumor-specific fluorescent antibodies. (Bouvet, M., and Hoffman, R. M. Glowing tumors make for better detection and resection. Sci. Transl. Med. 3, 110sf10, 2011).) Because tumors of all types express telomerase, the genetic labeling method that uses a telomerase-dependent adenovirus to deliver GFP specifically to tumors offers the potential for widespread application. (Kishimoto, H., et al. In vivo internal tumor illumination by telomerase-dependent adenoviral GFP for precise surgical navigation. Proc. Natl. Acad. Sci. USA, 106, 14514-17 (2009); Kishimoto, H., et al. Selective metastatic tumor labeling with green fluorescent protein and killing by systemic administration of telomerase-dependent adenoviruses. Molecular Cancer Therapeutics 8, 3001-08 (2009); Kishimoto, H., et al. Tumor-selective adenoviral-mediated GFP genetic labeling of human cancer in the live mouse reports future recurrence after resection. Cell Cycle 10, 2737-41 (2011).) This genetically-stable labeling method also allows detection of cancer recurrence, which is potentially a great advantage. However, the viral vector must be further tested for safety in humans before this method can be translated. (Fujiwara, T., et al. Phase I trial of intratumoral administration of OBP-301, a novel telomerase-specific oncolytic virus, in patients with advanced solid cancer. Evaluation of biodistribution and immune response. J. Clin. Oncol. 26, 3572, 2008).)

FGS can enable successful cancer surgery in many instances where bright-light surgery (BLS) cannot. There are three important issues regarding the clinical application of FGS: 1) proper tumor labeling; 2) a simple, portable imaging system for the operating room; and 3) patient-like mouse models in which to develop the technology. Although techniques for tumor fluorescence labeling and patient-like animal models are widely described in the prior art, the fixed light source/detector systems used to image the fluorescent label are impractical for clinical use for the reasons described above, and others. This invention fulfills the need, not met by prior art systems, for a method utilizing a simple but powerful, portable, real-time imaging system for FGS.

Exceptional results are obtained with the portable imaging systems described in the various embodiments of the invention. Hand-held probes are very lightweight. Portable fluorescence imaging systems are relatively inexpensive and are available for less than $1,000. Conversely, fixed imaging systems typically have sold for between $25,000 and $200,000. In addition, the portable imaging systems out-perform the larger, more cumbersome, highly expensive imaging systems which are impractical for use in the hospital operating room and other clinical settings. Thus, the disclosed method and various embodiments of using the portable system makes possible the widespread, revolutionary use of FGS to treat cancer and other conditions.

Embodiments of the present invention include a system and method for using a portable digital fluorescence imaging device for fluorescence-guided surgery including fluorescence-guided surgery of cancer. Generally, the system and method include the use of a portable probe directed toward a surgical field to excite a fluorescent label-containing tissue within the surgical field and display a real-time image visualization of the surgical field on a display. This invention overcomes the aforementioned and other difficulties encountered with using prior art systems, like a fixed fluorescence imaging system. It is designed to facilitate widespread intraoperative and clinical real-time imaging of fluorescence-labeled primary and metastatic tumors, in both clinical and experimental applications. The widespread availability of real-time fluorescence imaging will aid researchers, surgeons, and other health care professionals in the diagnosis and treatment of human patients with cancer. This invention broadens the availability of FGS over that possible with other fixed imaging systems to both laboratory researchers and clinicians.

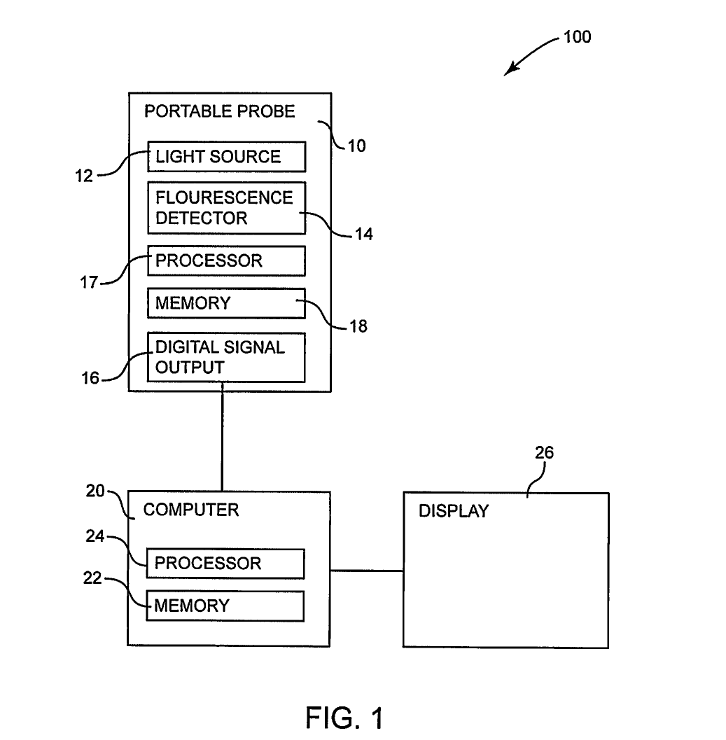

FIG. 1 is a schematic view of a portable digital imaging system 100 for FGS. The system 100 includes a portable probe 10 for real-time digital imaging of tissues containing fluorescent-labeled cells. The portable probe 10 comprises a light source 12, a fluorescence detector 14, and a digital signal output 16. The portable probe 10 may be of reasonable size and weight, and may be designed to be held in the operator's hand. To avoid tissue damage, the wavelength emitted by the light source 12 must be in the visible or near-infrared spectrum. In an embodiment utilizing GFP or a related fluorescent protein(s) as the fluorescent label, the required excitatory light source 12 is typically within the visible blue-light range and completely harmless to exposed tissue. The excitatory light source 12 excites the fluorescent label-containing tissue within the surgical field in response to the light source 12 of the portable probe 10 being directed toward the fluorescent label-containing tissue. This causes any fluorescent label within the field illuminated by the portable probe 10 to emit light. The light emitted from the fluorescent label is detected by the fluorescence detector 14 contained within the portable probe 10.

The portable probe 10 also includes a memory and processor, wherein firmware is stored on the memory and executed by the processor to convert the detected photons into a digital signal. In at least this way, the portable probe 10 converts the detected photons into a digital signal. Once a digital signal is created, the digital signal is sent from the portable probe 10 to a computer 20 through the digital signal output 16. The computer 20, having a memory 22 and a processor 24, operates software stored on the memory 22 by use of the processor 24 to process the digital signal sent from the portable probe 10 and displays on a screen 26 a real-time image visualization of the surgical field with the fluorescent label-containing tissue. The surgeon or other person practicing the invention then observes the displayed real-time image visualization of the surgical field on the screen in response to use of the portable probe 10. The display may be a standard video monitor, such as a computer screen or other video monitor screen type which is now commonly used in operating rooms for videoendoscopic surgery, other applications within the operating room and other clinical areas, or in the research lab and other experimental applications.

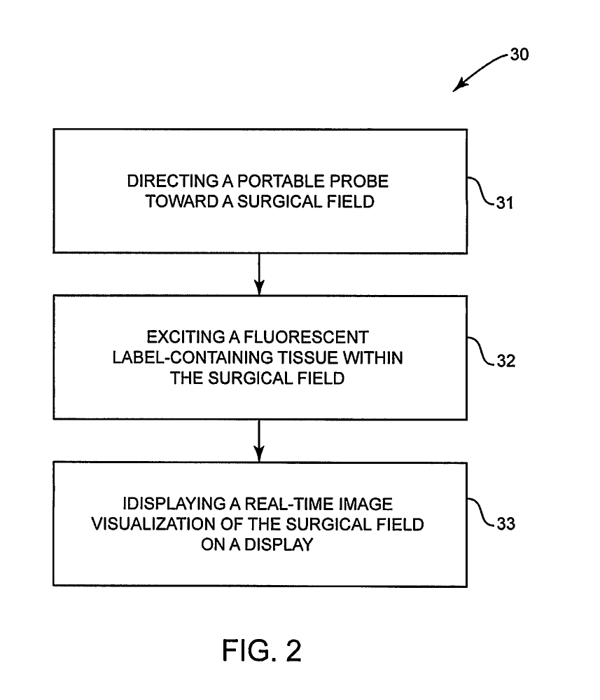

FIG. 2 is a flowchart of a method 30 of using portable digital imaging system for FGS. The method 30 includes Step 31 of directing a portable probe toward a surgical field, wherein the probe comprises a light source, a fluorescence detector, and a digital signal output. After Step 31, the method 30 includes Step 32 of exciting a fluorescent label-containing tissue within the surgical field in response to the light source of the probe illuminating the tissue. The method 30 then includes Step 33 of displaying a real-time image visualization of the surgical field on a screen in response to a computer in communication with the monitor receiving image data of the fluorescent label-containing tissue from the digital signal output of the portable probe.

The method 30 may further include preoperative labeling of the desired cell(s) using (one or more) fluorescent probes. In one embodiment of the invention, the fluorescent probe is GFP, however this is illustrative and not meant to be limiting. Other fluorescent labels may be used with other embodiments of the invention.

After preoperative labeling of the target cells, the method 30 may be utilized intraoperatively as previously described. The surgical field is prepared to expose the tissue, organs, and the overall region of interest for imaging. The portable probe is then brought into the sterile field and method 30 utilized. The probe itself may be sterile, encased in a sterile plastic sleeve, or otherwise prepared for use in surgery without breaking sterility of the operation or violating other standard care practices.

The method 30 may further include resecting the fluorescent label-containing tissue. The method 30 may include moving the portable probe to direct light from the light source to various fluorescent label-containing tissues within the surgical field. Further, the method may also include directing light from the light source at various angles onto the surgical field in order to display a desired image of the fluorescent label-containing tissue for better visualization of the fluorescent label-containing tissue for resection. It will be understood that method 30 may be utilized in an operating room by a surgeon, in a clinic or other clinical settings, or in the research lab and other experimental applications.

The method 30 may be practiced in cancer-patient-like mouse models. Previous development of FGS has shown enhanced resection of human tumors in orthotopic mouse models and greatly improved outcomes compared to BLS, including disease-free survival, with the use of fixed-fluorescence imaging systems. The method 30 and portable probe 10 eliminate the aforementioned difficulties with using a fixed-fluorescence imaging system, even in a laboratory setting with small experimental animal subjects. The portable probe 10 utilized in method 30 is less cumbersome and easier to use then the much larger, bulkier fixed system. Not only is a portable probe 10 very convenient to handle versus the corresponding use of a fixed fluorescence imaging system, but the readout is directly on a computer screen so that no special filters or filter goggles are needed. Finally, background autofluorescence is reduced with use of the portable probe 10.

Method 30 may be practiced using colon-cancer-patient tumor specimens initially established subcutaneously in NOD/SCID mice immediately after surgical resection from a human patient. The patient tumors are harvested from the NOD/SCID human and transplanted orthotopically into nude mice. Eight weeks after orthotopic implantation of the human colon cancer tumor grafts, a monoclonal anti-CEA antibody, conjugated with a fluorophore, such as, but not limited to, Alexa 448, is delivered to the tumor-bearing mice as a single intravenous dose 24 hours before laparotomy. Resection of the primary colon tumor is performed using the portable probe 10. During FGS following the processes of method 30, the primary tumor is clearly visible on a display.

Using a portable probe in FGS of orthotopic colon cancer in the nude mouse model is by way of example only. Method 30 is intended to be used with other cell types forming solid tumors, and in both experimental animals and humans. Method 30 may be used to aid in complete resection of the primary tumor or metastasis. Additionally, method 30 may be used to identify the presence and location of tumor metastases, either intraoperatively or transcutaneously.

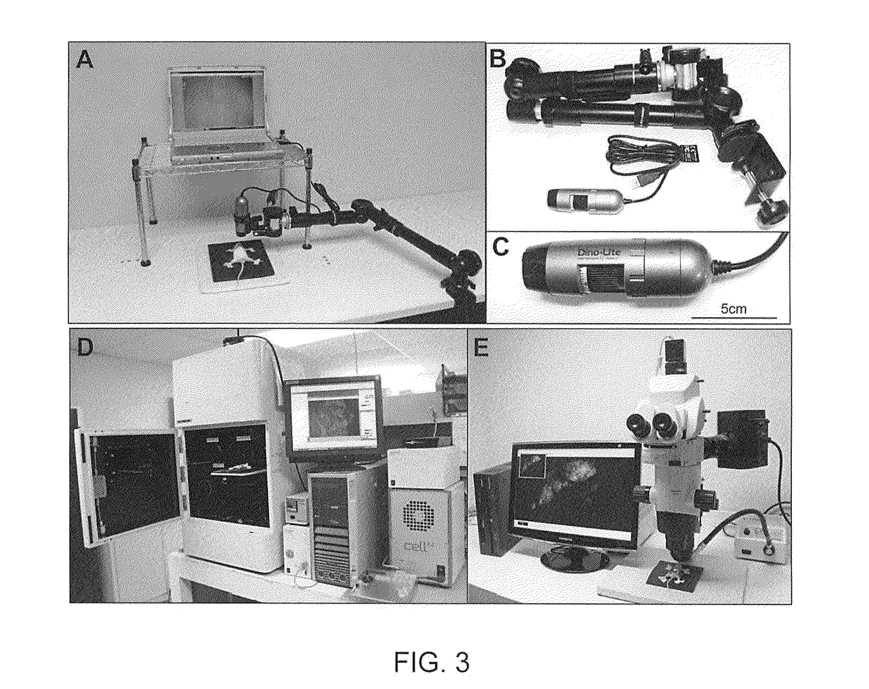

FIG. 3 is a series of photographs demonstrating an example of a portable imaging system for use in FGS contrasted with two corresponding fixed imaging systems. (A) View of the Dino-Lite portable imaging system with a Dino-Lite digital camera. (B) The Dino-Lite portable imaging system. (C) Close-up view of a Dino-Lite digital camera. The camera's dimensions are 10.5×3.2 cm and the weight is only 105 g. (D) View of the Olympus OV100 Small Animal Imaging System. (E) View of the Olympus MVX10 Macro View.

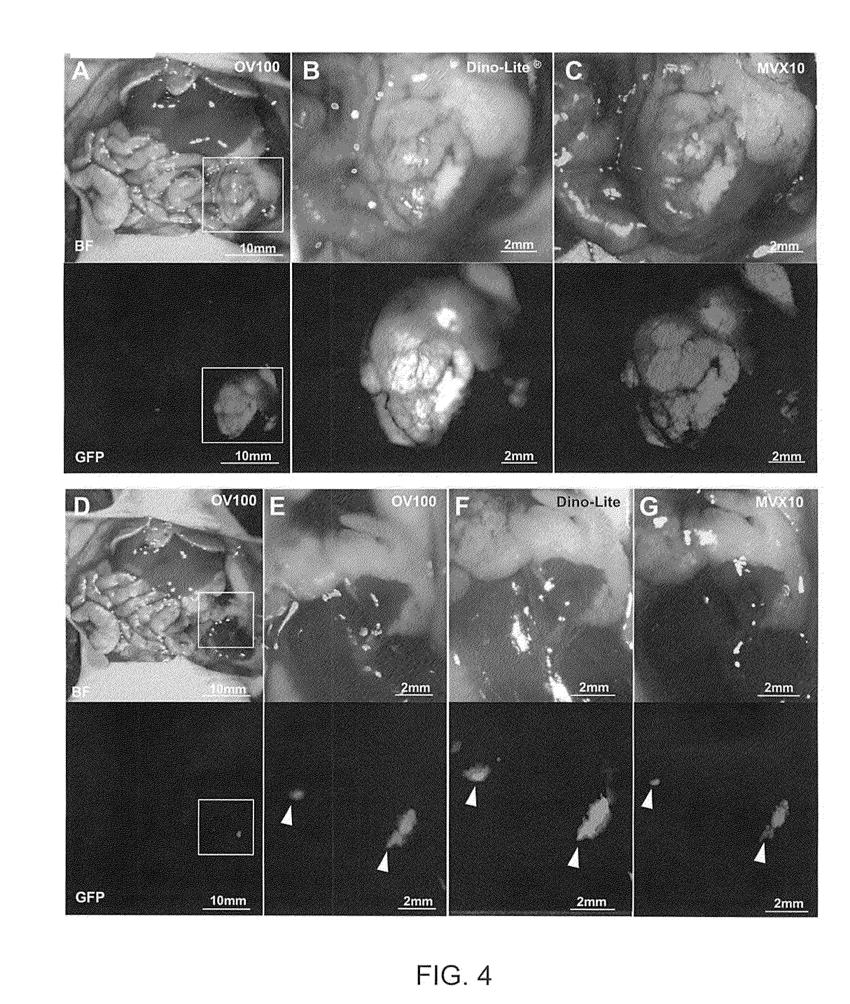

FIG. 4 is images of genetically-labeled MiaPaCa-2-GFP tumor using an example portable imaging system for use in FGS, contrasted with images taken from two corresponding fixed imaging systems. The primary MiaPaCa-2-GFP tumor was imaged with the OV100 at a magnification of ×0.14 (A), the Dino-Lite at a magnification of ×30 (B) and the MVX10 at a magnification of ×1.6 (C). The residual tumor after BLS was imaged with the OV100 at a magnification of ×0.14 and ×0.56 (D and E, respectively), the Dino-Lite at a magnification of ×30 (F), and the MVX10 at a magnification of ×1.6 (G). Boxes in (A) indicate the view areas of (B) and (C), and boxes in (D) indicate the view areas of (E), (F) and (G). White arrowheads indicate the detected residual tumors after bright—light surgery (“BLS”). It is understood that these images are an example of the present invention and is in no way considered to limit the scope of the claims.

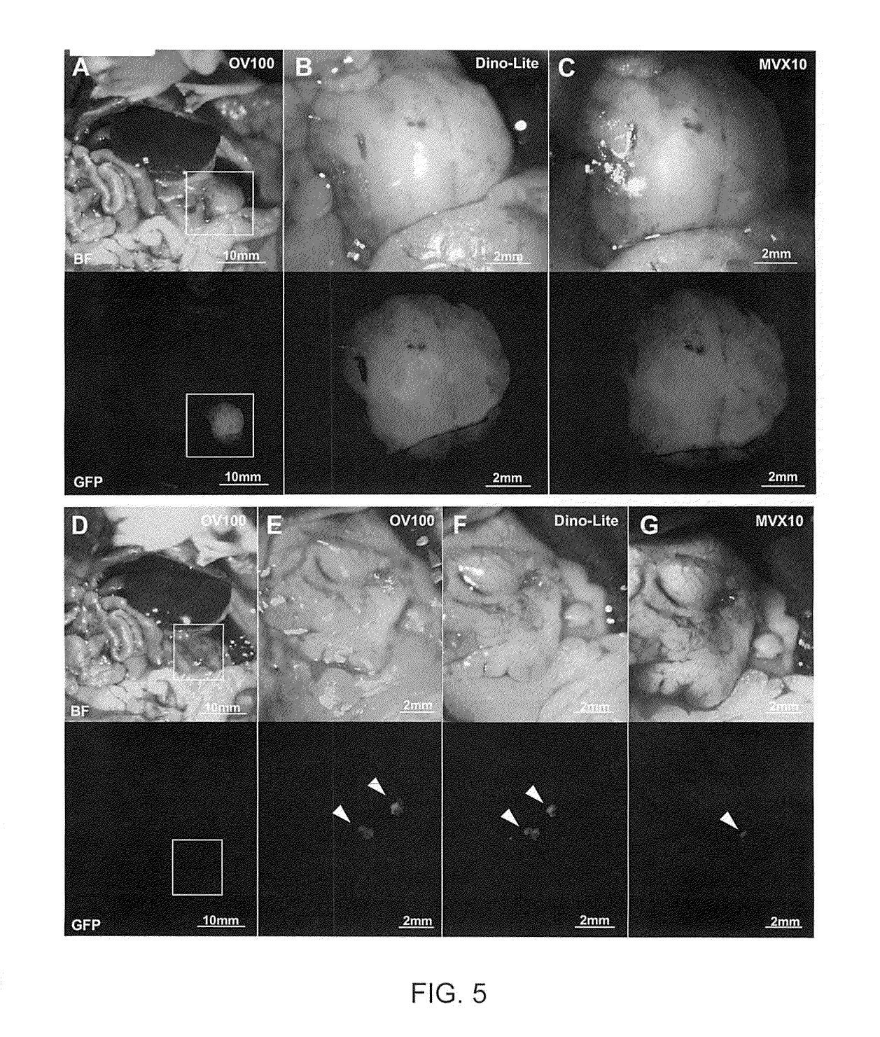

FIG. 5 is images of the BxPC3 tumor labeled with fluorescent anti-CEA antibody using an example portable imaging system for use in FGS, contrasted with images taken with two corresponding fixed imaging systems. The primary BxPC3 tumor labeled with fluorescent anti-CEA antibody was imaged with the OV100 at a magnification of ×0.14 (A), the Dino-Lite at a magnification of ×30 (B) and the MVX10 at a magnification of ×1.6 (C). The residual tumor after BLS was imaged with the OV100 at a magnification of ×0.14 and ×0.56 (D and E), the Dino-Lite at a magnification of ×30 (F) and the MVX10 at a magnification of ×1.6 (G). Boxes in (A) indicate the view areas of (B) and (C), and boxes in (D) indicate the view areas of (E), (F) and (G). White arrowheads indicate the detected residual tumors after BLS. It is understood that these images are an example of the present invention and is in no way considered to limit the scope of the claims.

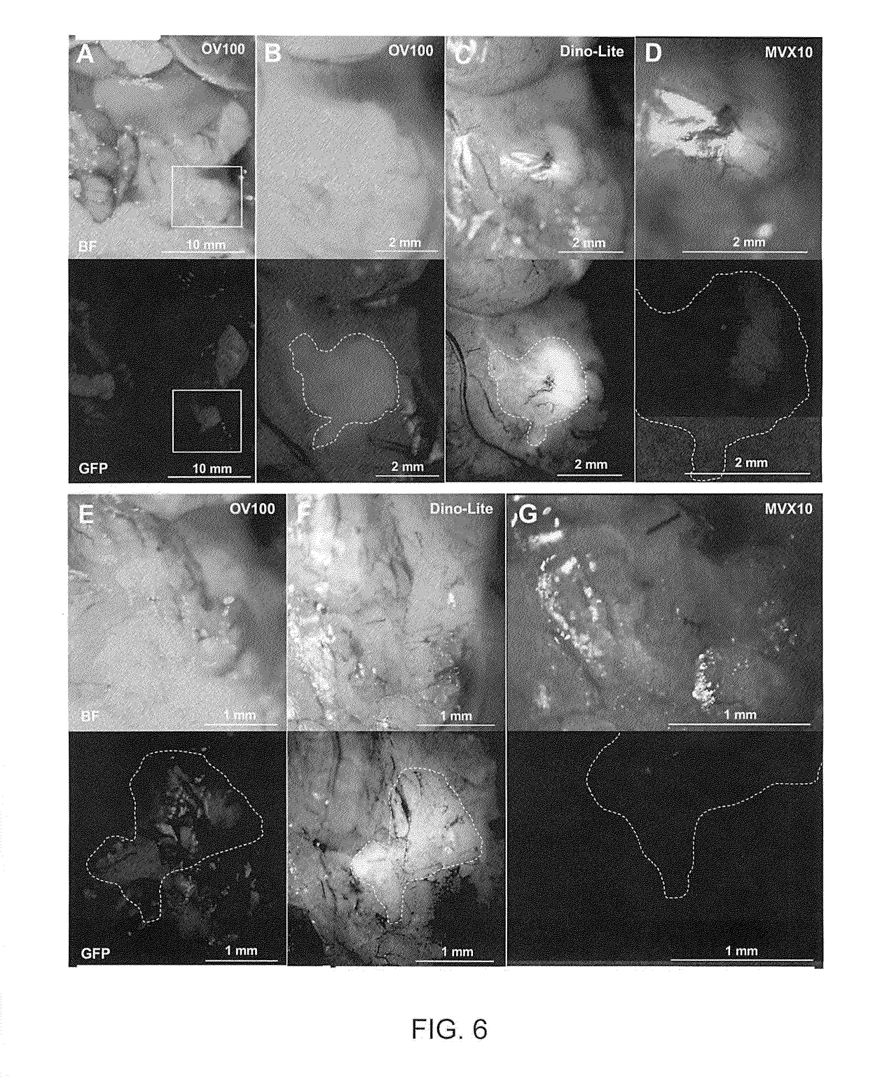

FIG. 6 is images of the pancreatic PDOX® tumor labeled with fluorescent anti-CA19-9 antibody using an example portable imaging system for use in FGS contrasted with images taken with two corresponding fixed imaging systems. The primary PDOX® tumor, labeled with fluorescent anti-CA19-9 antibody, was imaged with the OV100 at a magnification of ×0.14 and ×0.56 (A and B), the Dino-Lite at a magnification of ×30 (C) and the MVX10 at a magnification of ×1.6 (D). The MVX10 at a magnification of ×3.2 did not detect any signals from the residual tumor (G). The OV100 at a magnification of ×0.89 could not distinguish the residual tumor from background (E). The Dino-Lite at a magnification of ×50 could clearly distinguish the residual tumor from background (F). Boxes in (A) indicate the view areas of (B), (C) and (D). The areas surrounded by white broken lines indicate the estimated residual tumor areas after BLS. It is understood that these images are an example of the present invention and is in no way considered to limit the scope of the claims.

Postoperatively, the surgical resection bed can be imaged with a portable or fixed imaging system to further assess and confirm completeness of the surgical resection. Additionally, histologic evaluation of the resected specimen can be undertaken to confirm the absence of tumor cells in the tumor margins.

In yet another embodiment of the invention, mice undergo FGS using the portable probe 10 two weeks after orthotopic implantation of human colon cancer cells. Mice are anesthetized and their abdomens are sterilized. The intestine is delivered through a midline incision and FGS of the primary colon tumor using method 30 is performed using the portable probe 10 coupled to a display, wherein real-time imaging is produced using the portable device 10 in accordance with method 30 to guide resection of the tumor.

In another embodiment of the invention, portable noninvasive fluorescence imaging is used to assess tumor recurrence and progression. To assess for recurrence longitudinally and to follow tumor progression postoperatively, weekly noninvasive imaging of nude mice is performed following resection of human solid cancer xenografts. The entire animal is externally examined with the portable probe and recurrent tumors are identified by real-time visualization on the monitor.

An additional embodiment of the invention uses a GFP label created by introducing and selectively activating the GFP gene in malignant tissue in vivo by the use of OBP-401, a telomerase-dependent, replication-competent adenovirus expressing GFP. This powerful adjunct to surgical navigation has been demonstrated in nude mouse models that represent difficult surgical challenges—the resection of widely disseminated cancer. An HCT-116 human colon cancer lane is used as one example of a model of intraperitoneally disseminated human colon cancer and labeled by injecting the virus into the peritoneal cavity. Only the malignant tissue will fluoresce brightly. In the nude mouse intraperitoneal model of disseminated cancer, portable FGS enables resection of all tumor nodules labeled with GFP by OBP-401. The hand-held probe component of the portable digital imaging system used in this and other embodiments generates substantially less auto fluorescence compared to powerful fixed imaging systems in current use, increasing the contrast between cancerous and normal tissue resulting in a more accurately guided resection.

Still another embodiment of the invention is practiced where the fluorescent label is a fluorescent antibody. In an example of this embodiment using a human colon cancer cell type, patient colon tumors are initially established subcutaneously in NOC/SCID mice immediately after surgery. The tumors are then harvested from NOD/SCID mice and passed orthotopically in nude mice to make patient-derived orthotopic xenograft (PDOX®) models. Eight weeks after orthotopic implantation, a monoclonal anti-CEA antibody conjugated with Alexa 488 is delivered to the PDOX® models as a single intravenous dose 24 hours before laparotomy. A handheld portable fluorescence imaging device with a digital signal output attached to a monitor for real-time visualization of the fluorescent label is used. The portable system was easier to use, the procedure was accomplished in less time, and with less background auto fluorescence to complicate the distinction between labeled and unlabeled tissue than with a corresponding fixed imaging system. In this embodiment, the primary tumor is clearly visible at laparotomy when practicing the invention.

The embodiments and examples set forth herein were presented in order to best explain the present invention and its practical application and to thereby enable those of ordinary skill in the art to make and use the invention. However, those of ordinary skill in the art will recognize that the foregoing description and examples have been presented for the purposes of illustration and example only. The description as set forth is not intended to be exhaustive or to limit the invention to the precise form disclosed. Many modifications and variations are possible in light of the teachings above.

What is claimed is:

1. A method for use of a portable imaging device for fluorescence-guided surgery comprising:
directing a portable probe toward a surgical field, wherein the probe comprises a light source, a fluorescence detector, and a digital signal output;
exciting a fluorescent label-containing tissue within the surgical field in response to the light source of the portable probe illuminating the fluorescent label-containing tissue; and
displaying a real-time image visualization of the surgical field on a display in response to a computer in communication with the display receiving image data of the fluorescent label-containing tissue from the digital signal output of the portable probe.

2. The method of claim 1, further comprising labeling desired tissue with a fluorescent label.

3. The method of claim 1 wherein the fluorescent label is a fluorophore.

4. The method of claim 1 wherein the fluorescent label is a fluorescent protein.

5. The method of claim 1 wherein the fluorescent label is a fluorescent antibody.

6. The method of claim 1 wherein the portable probe is a portable digital microscope.

7. The method of claim 1, further comprising performing treatment of cancer.

8. The method of claim 7, wherein performing treatment of cancer includes resecting a human tumor.

9. The method of claim 1, wherein displaying the real-time image visualization comprises operating the fluorescence detector to detect photons emitted by the fluorescent label-containing tissue in response illuminating the fluorescent label-containing tissue.

10. The method of claim 9, further comprising converting the detected photons into a digital signal in response to operation of the portable probe.

11. The method of claim 10, further comprising sending the digital signal through the digital signal output of the portable probe to a computer to process the signal and display the real-time image visualization of the surgical field on the display.

12. A portable digital imaging system for fluorescence-guided surgery, the system comprising:
a portable probe comprising a light source, a fluorescence detector, a digital signal output, a memory and a processor;
a computer in communication with the portable probe, the computer comprising a memory and a processor; and
a display, wherein:
the portable probe excites a fluorescent label-containing tissue within a surgical field in response to the light source of the portable probe illuminating the fluorescent label-containing tissue; and
the computer displays a real-time image visualization of the surgical field on the display in response to the computer in communication with the display receiving and processing a digital signal from the digital signal output of the portable probe.

13. The system of claim 12, further comprising a fluorescent label, wherein the desired tissue is labeled with the fluorescent label.

14. The system of claim 12, wherein the portable probe is a portable digital microscope.

15. The system of claim 12, wherein the light source of the portable probe operates within a predetermined wavelength.

16. The system of claim 15, wherein the predetermined wavelength of the light source is determined by the wavelength required to excite the fluorescent label-containing tissue and not damage surrounding tissue.

17. The system of claim 12, wherein the fluorescence detector of the portable probe detect photons emitted by the fluorescent label-containing tissue in response illuminating the fluorescent label-containing tissue.

18. The system of claim 17, wherein the portable probe converts the detected photons into a digital signal in response to operation of the portable probe.

19. The system of claim 18, wherein the digital signal output sends the digital signal to the computer.

20. The system of claim 19, wherein the computer processes the digital signal and sends the processed signal to the display for displaying the real-time image visualization of the surgical field.
